(19) United States
(12) Patent Application Publication
(10) Pub. No.: US 2013/0211185 A1
(43) Pub. Date: Aug. 15, 2013

(54) Cushioned Resilient Intravaginal Urinary Incontinence Device and Method of Making Same

(71) Applicants: Raymond J. Hull, JR., Hampton, NJ (US); Rainer Armbruster, Wolfach (DE)

(72) Inventors: Raymond J. Hull, JR., Hampton, NJ (US); Rainer Armbruster, Wolfach (DE)

(73) Assignee: MCNEIL-PPC, INC., Skillman, NJ (US)

(21) Appl. No.: 13/881,838

(22) PCT Filed: Jun. 1, 2012
(86) PCT No.: PCT/US2012/040392

§ 371 Date: Apr. 26, 2013

Related U.S. Application Data
(60) Provisional application No. 61/492,845, filed on Jun. 3, 2011.

(51) Int. Cl. B29C 45/16; A61F 2/00

(52) U.S. Cl. CPC B29C 45/1675; A61F 2/0022
USPC 600/29; 264/255

(57) ABSTRACT

A method of making an injection molded device includes the steps of (a) assembling a mold comprising a primary male mold half and a primary female mold half to define a primary mold cavity; (b) injecting a first fluid polymeric material into the primary mold cavity and permitting the first fluid polymeric material to solidify to form a device frame; (c) replacing the primary female mold half with a secondary female mold half; (d) restraining the device frame in the resulting mold cavity and injecting a second fluid polymeric material to form a first portion of an outer layer that retains the shape of the first overmold cavity; (e) replacing the primary male mold half with a secondary male mold half; and (f) injecting an additional amount of the second fluid polymeric material into the resulting cavity to form a sufficiently integral outer layer about the device frame.

This application is the national stage filing under 35 USC 371 of international application PCT/US2012/040392 filed on Jun. 1, 2012, which claims the benefit of U.S. provisional application 61/492,845 filed on Jun. 3, 2011, the complete disclosures of which are hereby incorporated herein by reference for all purposes.

### FIELD OF THE INVENTION

The present invention relates to an intravaginal urinary incontinence device. More specifically, this invention relates to a method of making a device that has a working portion and an anchoring portion and is overmolded with a cushioning material. The device is very useful for reducing or preventing urinary incontinence, and the overmolded cushioning material reduces the potential for vaginal irritation by reducing the pressure applied by the device on the vaginal wall during insertion, use or removal.

### DESCRIPTION OF THE PRIOR ART

Stress urinary incontinence is a problem for many women. It is characterized by leakage of urine during a stressing event, such as a cough or a sneeze. Many devices have been designed to reduce or prevent stress urinary incontinence. Tutrone, Jr., U.S. Pat. No. 5,603,685 relates to inflatable devices and a means to provide a device that is small for insertion into the vagina and enlarges to a required shape and pressure to reduce or prevent urinary incontinence. Zunker et al., U.S. Pat. No. 6,090,098 relates to tampon-like devices, each made with a combination of absorbing and/or non-absorbing fibrous materials. Ulmsten et al., U.S. Pat. No. 6,645,137 relates to a coil that expands in the vagina to support the urinary system. Biswas, U.S. Pat. No. 5,036,867 relates to a compressible resilient pessary. James, U.S. Pat. No. 6,460,542 relates to a specifically shaped rigid pessary.

More recent developments have attempted to provide stent-like supports for deployment into the vagina. For example, Bartning et al., US Pat. App. Nos. 2008/0033230 and 2008/0009662 relate to an intravaginal urinary incontinence device that has an anchoring portion and a working portion. These documents also disclose covering the structure with a biocompatible material. In addition, there are numerous patents that relate to the use of small, appropriately-sized stents that are designed to keep body passages.

Sinai et al., US Pat. App. No. 2008/0281149, relates to an incontinence device with an internal and/or external resilient support member that biases arms of the incontinence device.

Finally, Ziv et al., WO2008/010214, relates to an intravaginal apparatus for treating urinary incontinence having a node connecting a support section and an anchoring section. This discloses the use of arms for the support and/or anchoring section made of silicone, nylon, polyurethane, foam polystyrene, metal, or an over molding of two materials.

Several of these references have begun to recognize the potential for the support structures to irritate vaginal tissues, and they have enclosed structural elements in tubing or other outer layers. Alternatively or in addition to this, bag-like covers have been suggested. Unfortunately, these developments have not fully addressed all of the issues relating to cushioning, comfort and product reliability.

Therefore, there are continuing needs for improved intravaginal urinary incontinence devices that can effectively reduce or prevent urinary incontinence on the one hand and can also provide appropriately located cushioning to avoid increased risk of vaginal damage. Further, there are continuing needs for the improved manufacture of safe and inexpensive intravaginal urinary incontinence devices.

### SUMMARY OF THE INVENTION

We have found novel methods for the improved manufacture of safe and inexpensive intravaginal urinary incontinence devices.

In one embodiment, a method of making an injection molded device includes the steps of (a) assembling a mold comprising a primary male mold half and a primary female mold half to define a primary mold cavity; (b) injecting a first fluid polymeric material into the primary mold cavity and permitting the first fluid polymeric material to solidify to form a device frame; (c) removing the primary female mold half and replacing it with a secondary female mold half to provide a first overmold cavity; (d) restraining the device frame in the first overmold cavity and injecting a second fluid polymeric material into the first overmold cavity and permitting the second fluid polymeric material to solidify to sufficiently to form a first portion of an outer layer that retains the shape of the first overmold cavity; (e) removing the primary male mold half and replacing it with a secondary male mold half to provide a second overmold cavity; and (f) injecting an additional amount of the second fluid polymeric material into the second overmold cavity and permitting the second fluid polymeric material to unite with the first portion of the outer layer to form a sufficiently integral outer layer about the device frame. The first overmold cavity is defined by the primary male mold half, the secondary female mold half, and the device frame, and the second overmold cavity is defined by the secondary male mold half, the secondary female mold half, the device frame, and the first portion of the outer layer.

In an alternative embodiment, a method of making an injection molded intravaginal urinary incontinence device includes the steps of (a) assembling a mold comprising a primary male mold half and a primary female mold half to define a primary mold cavity; (b) injecting a fluid high modulus polymeric material into the primary mold cavity and permitting the high modulus polymeric material to solidify to form a device frame; (c) removing the primary female mold half and replacing it with a secondary female mold half to provide a first overmold cavity; (d) restraining the device frame in the first overmold cavity and injecting a fluid cushioning polymeric material into the first overmold cavity and permitting the cushioning polymeric material to solidify to sufficiently to form a first portion of an outer layer that retains the shape of the first overmold cavity; (e) removing the primary male mold half and replacing it with a secondary male mold half to provide a second overmold cavity; and (f) injecting an additional amount of the fluid cushioning polymeric material into the second overmold cavity and permitting the cushioning polymeric material to unite with the first portion of the outer layer to form a sufficiently integral outer layer about the device frame. The first overmold cavity is defined by the primary male mold half, the secondary female mold half, and the device frame, and the second overmold cavity is defined by the secondary male mold half, the secondary female mold half, the device frame, and the first portion of the outer layer.

### BRIEF DESCRIPTION OF THE DRAWING

FIG. 1 is a front plan view of a device according to the present invention.

FIG. 2 is a perspective view of an overmolded device frame according to one embodiment of the present invention.

FIG. 3 is a perspective view of a two-part injection mold assembly according to the present invention.

FIGS. 3A and 3B are schematic cross-sections of the two-part injection mold assembly of FIG. 3 in the region of the mold cavity.

FIG. 4 is a perspective view of the two-part injection mold assembly of FIG. 3 with a primary female mold half replaced with a secondary female mold half.

FIGS. 4A and 4B are schematic cross-sections of the two-part injection mold assembly of FIG. 4 in the region of the mold cavity.

FIG. 5 is a perspective view of the two-part injection mold assembly of FIG. 3 with both primary male and female mold halves replaced with secondary male and female mold halves.

FIGS. 5A and 5B are schematic cross-sections of the two-part injection mold assembly of FIG. 5 in the region of the mold cavity.

FIG. 6 is an exploded perspective view of the two-part injection mold assembly of FIG. 4.

FIG. 7 shows the perspective view of the two-part injection mold assembly of FIG. 4 with a first set of plugs mounted on a first plug mounting plate.

FIG. 8 shows the perspective view of the two-part injection mold assembly of FIG. 5 with a second set of plugs mounted on a second plug mounting plate.

FIG. 9 is a perspective view of a device frame produced in the present invention.

FIG. 10 is a perspective view of the device frame of FIG. 9 with an outer layer portion overmolded thereon—an intermediate step of the method of the present invention.

FIG. 11 is a perspective view of the device frame of FIG. 9 fully overmolded with the cushioning material produced according to the method of the present invention.

### DETAILED DESCRIPTION OF THE PREFERRED EMBODIMENTS

We have discovered that the descriptions of how to protect structural elements of intravaginal incontinence devices with a softer material disclosed in the art fail to show how to manufacture commercial quantities of inexpensive devices with adequate comfort for the user. First, we have not found overmolding processes with adequate control of the process to provide cushioning where needed on small diameter structures without creating unnecessary bulk. Unnecessary bulk can make it difficult and/or impossible to provide a small enough applicator for the intravaginal incontinence device for comfortable insertion into the vagina with enough expansion to provide necessary support to an associated urinary system.

During the development of this invention, we have also discovered that low-cost injection molded structural elements in intravaginal incontinence devices can have a rough edge or part line at the periphery of the mold portions. This has the potential to irritate the vagina. Covering this device in a bag did not adequately address this problem, as these rough edges simply tore the bag during packaging of the product into an applicator and/or during the expulsion of the device to deploy it into a vagina.

Further, we have discovered that some materials used in the manufacture of intravaginal urinary incontinence devices may be susceptible to deterioration if exposed to processing compositions and/or the environment.

Therefore, we have developed a controlled process to fully overmold the device to answer some of these problems. This overmolded material can be non-uniform about the structural element that it covers. For example, the overmold material can be biased in a manner that the structural element is not located in the center of the overmold material. This will be discussed in greater detail, below.

It will be recognized that overmolding the structural elements of an intravaginal incontinence device increases the contact area between the device and the user's body tissue that it may engage, reducing the pressure (force per unit area). This helps to reduce or minimize vaginal irritation during insertion, use or removal.

The intravaginal incontinence devices of the present invention have a working portion to provide support to an associated urinary system and an anchoring portion to hold the device in optimal position during use. These structural elements are additionally covered to cushion the body from irritation.

As used herein the specification and the claims, the term “stent” and variants thereof relate to a device used to support a bodily orifice, cavity, vessel, and the like. The stent is resilient, flexible, and collapsible with memory. The stent may be any suitable form, including, but not limited to, scaffolding, a slotted tube or a wire form.

As used herein the specification and the claims, the term “wire form” and variants thereof relate to a structure formed of at least one wire or wire-like material that is manipulated and optionally secured (e.g., by welding and/or molding) in a desired three-dimensional structure.

As used herein, the term “bearing surface” and variants thereof relate to certain portions of the device that bear on and apply pressure to the vaginal epithelium during the insertion, use and removal. The existence of bearing surfaces is significant, because poorly designed devices may have dangerous bearing surfaces that can damage the vagina and/or surrounding body tissue. This damage could include irritation, erythema, and weakened or even necrotic vaginal tissue. Therefore, it is critical to protect the vaginal epithelium by cushioning actual and potential bearing surfaces.

As used herein, the term “device interior” and variants thereof relate to the inner portions of the device, directed toward a longitudinal axis and away from the bearing surfaces that are capable of contacting the vaginal epithelium. The device interior also will be described with reference to the figures, below.

As used herein, the term “overmolding” and variants thereof relate to injection molding processes where the cushioning material is molded onto the device frame (i.e. the wire or stent). The overmolding is performed in such a manner that the cushioning material fully encapsulates the device frame. The use of primers or adhesives is not required to achieve an optimum bond between the device frame or structural elements and the overlying cushioning material.

As used herein, the term “cushioning material” and variants thereof relate to any material which is soft in nature, the cushioning portion of the device provides softness and comfort and helps to reduce or minimize vaginal irritation and pressure mounted by the device on the vaginal epithelium during insertion, use or removal.

The intravaginal incontinence devices of the present invention include a working portion and anchoring portion. These portions are the structural elements of the device (also referenced as the “device frame”). The working portion provides support to an associated urinary system, and the anchoring portion maintains the working portion in an optimal location for this support. The overlying cushioning material provides comfort to the user. It can both smooth out any rough edges resulting from parting lines in the mold that formed the device frame, and it can increase the surface area over which the device contacts the user's body tissue to reduce the pressure.

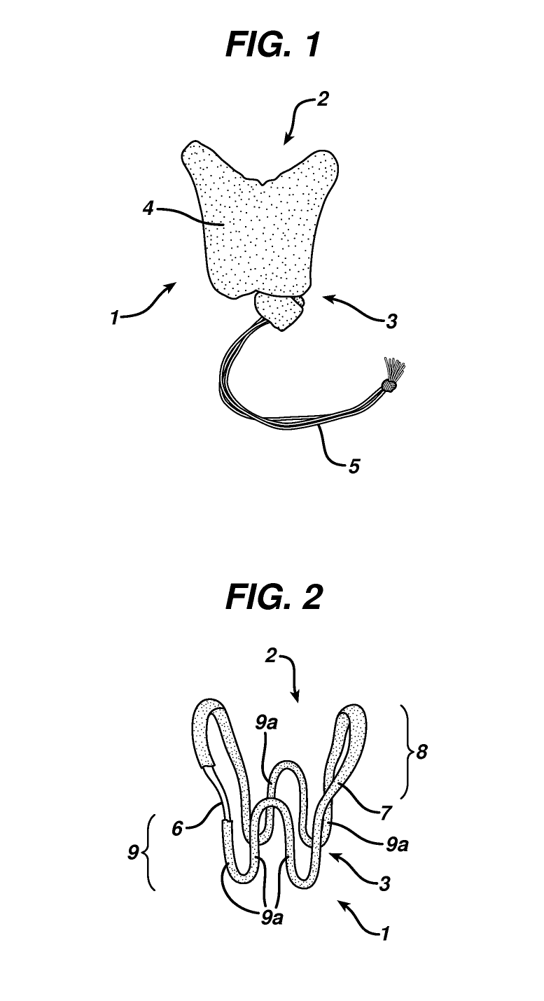

Suitable shapes of devices according to the present invention are taught in US Pat. App. Nos. 2008/0009664, and 2008/0033230, and 2008/0009662, the disclosures of which are hereby incorporated by reference in their entirety. Referring to FIGS. 1 and 2, there is shown a device 1 according to the present invention. The device 1a has an insertion end 2 and a withdrawal end 3. The device includes an outer flexible enclosure, such as a bag 4, substantially containing a resilient device frame, e.g., a stent, and having a withdrawal element, such as string 5.

In one embodiment, the flexible enclosure 4 contains a resilient device frame 6 covered with an overmolded outer layer 7 of cushioning material, such as shown in FIG. 2. The frame 6 includes an anchoring portion 8 that is disposed proximate the insertion end 2, and a working portion 9 that is disposed proximate the withdrawal end 3a. The working portion 9 has opposed faces defined by a plurality of substantially longitudinally oriented elongate elements, such as struts, 9a. Working portion 9 has an initial equivalent diameter d1 ranging from 20 mm to 170 mm and a length L₁ ranging from 15 mm to 60 mm. Where the working portion is non-cylindrical, the equivalent diameter is the maximum distance in millimeters between opposed faces.

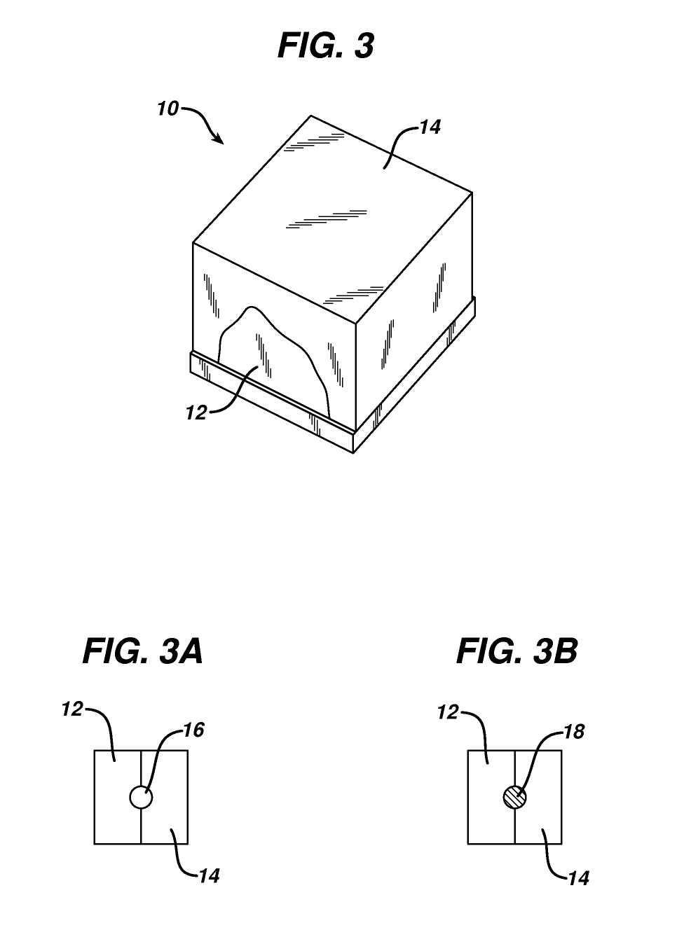

The method of the present invention will be discussed in reference to FIGS. 3-11 showing various configurations of a two-part mold used in a multistep molding system and intermediate products and final products formed in this system.

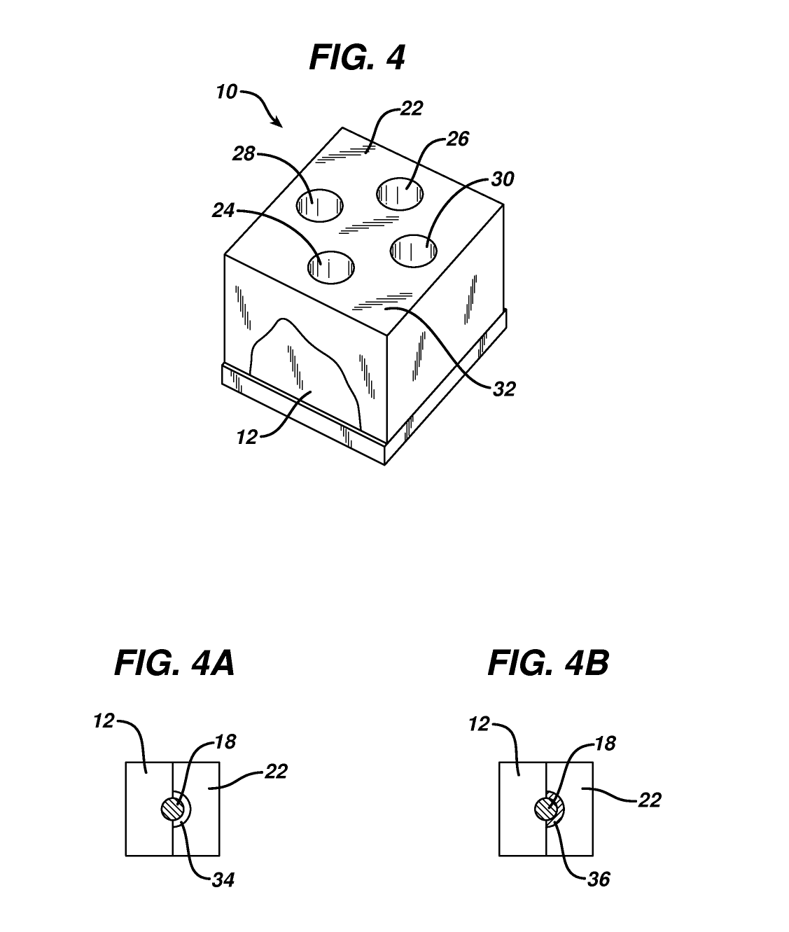

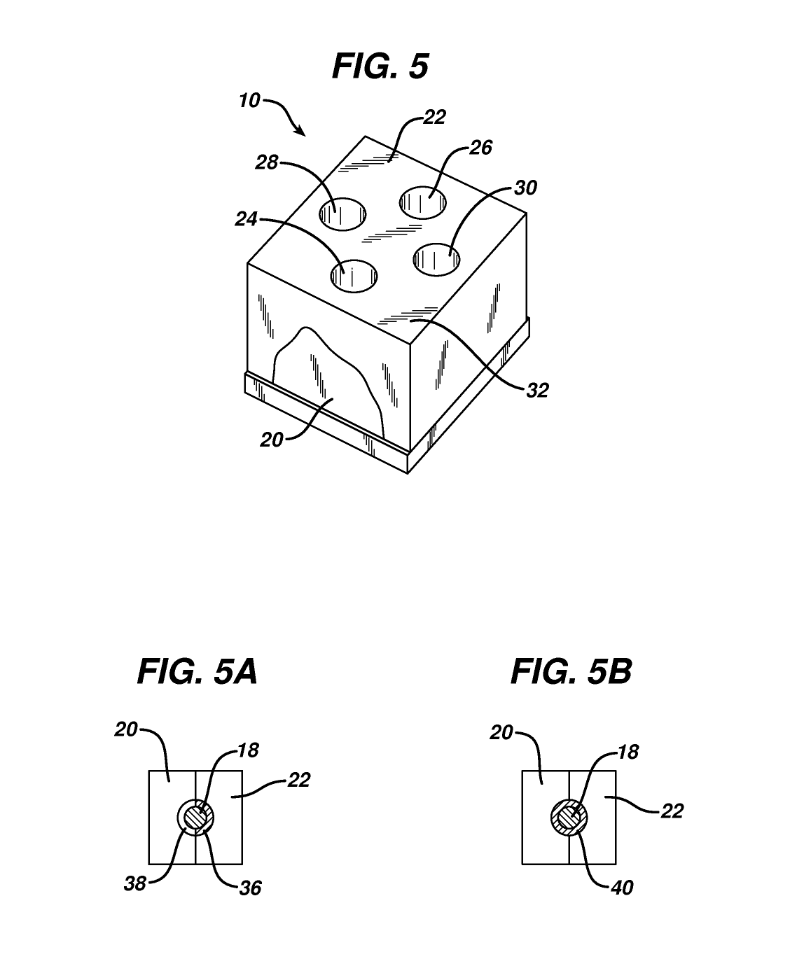

As shown in FIG. 3, the multistep molding system used in the present invention includes a two-part mold 10 with interchangeable male and female mold halves 12, 14. The first step, shown in FIG. 3 and FIGS. 3A and 3B (schematic cross-sections of the two-part mold 10 in the region of the primary mold cavity), employs a primary male mold half 12 and a primary female mold half 14 that together define a primary mold cavity 16 (FIG. 3A) dimensioned to form the device frame 18 (FIG. 3B). During this step, the material used to form the device frame is injected into the mold cavity. In the final molding step shown in FIG. 5, the primary male mold half 12 and primary female mold half 14 have been replaced with secondary male mold half 20 and secondary female mold half 22 that define a mold cavity dimensioned to form the fully overmolded device. An intermediate molding step, shown in FIG. 4, occurs using the primary male mold half 12 and the secondary female mold half 22.

Again, referring to FIG. 3 (including 3A and 3B), the primary male and female mold halves 12, 14 define a primary mold cavity 16. During this step, the material used to form the device frame is injected into the primary mold cavity 16. The device frame material solidifies to form the device frame 18, and the primary female mold half 14 is removed.

Referring to FIG. 4 (including 4A and 4B), it can be seen that the primary female mold half 14 has been replaced with the secondary female mold half 22 having a plurality of ports 24, 26, 28, 30 in a top, outer surface 32 thereof. In this step, the primary male mold half 12, the secondary female mold half 22, and the device frame 18 define a first overmold cavity 34, and the material used to form the outer layer is injected into the first overmold cavity 34. The outer layer material cools sufficiently to retain its shape as a portion of the outer layer 36, and the primary male mold half 12 is removed.

Referring to FIG. 5 (including 3A and 3B), it can be seen that the primary male mold half 12 has been replaced with the secondary male mold half 20. In this step, the secondary male mold half 20, the secondary female mold half 22, the device frame 18, and the first portion of the outer layer 34 define a second overmold cavity 38, and additional material used to form the outer layer is injected into the second overmold cavity 38. The additional material and the heat of the mold permits the outer layer material to unify to form a sufficiently integral outer layer 40 (shown in FIG. 5B).

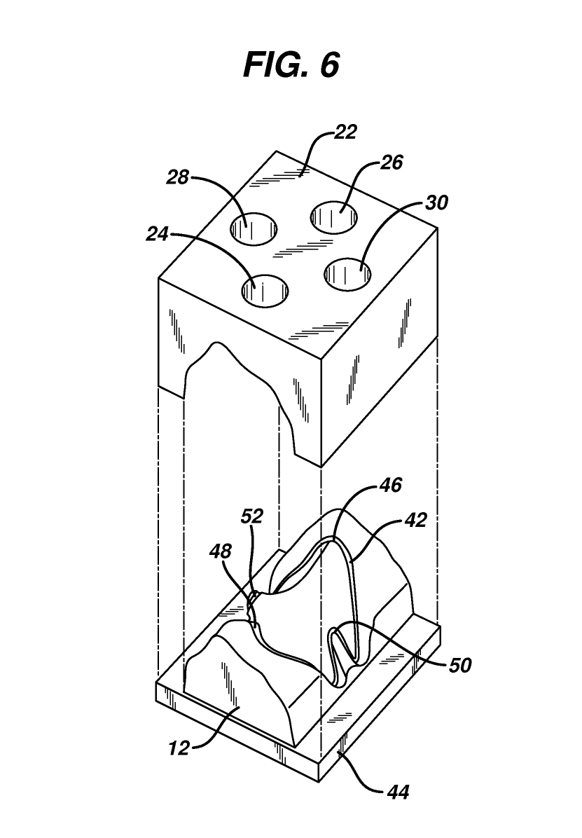

To make an incontinence device of the present invention, the male mold half is contoured with an inverted, relatively “V-shaped” cross-section and provides a channel 42 that forms a portion of the mold cavity. FIG. 6 shows an exploded perspective view of the two-part mold 10 of FIG. 4, including the primary male mold half 12 and the secondary female mold half 22. This perspective view shows a base 44 on which is mounted the primary male mold half 12, which has the inverted, relatively “V-shaped” cross-section. The channel 42 defines one continuous surface of the primary mold cavity 16. The primary male mold half 12 has additional material removed to permit the matching female mold halves (primary female mold half 14 and secondary female mold half 22) to interact to form the desired mold cavities, such as the primary mold cavity 16, first overmold cavity 34, and second overmold cavity 38 discussed above.

In greater detail, the secondary female mold half 22 has a channel (not specifically shown in full) that interacts with the channel(s) of the primary and secondary male mold halves 12 and 20 to form the desired mold cavities. In the embodiment shown in FIGS. 4-6, the secondary female mold half 22 has four ports 24, 26, 28, 30 arranged in a diamond-shaped pattern. These ports 24, 26, 28, 30 provide passages from the first and second overmold cavities 34, 38 to a source of overmold material (not shown). A first pair of these ports 24, 26 are disposed at opposite corners of the diamond, and they provide passages to upper portions of the mold cavity, corresponding to the top portion of curves 46, 48 shown in relation to the primary male mold half 12 in FIG. 6. A second pair of these ports 28, 30 are disposed at the remaining corners of the diamond, and they provide passages to lower portions of the mold cavity, corresponding to the top portion of curves 50, 52 shown in relation to the primary male mold half 12 in FIG. 6.

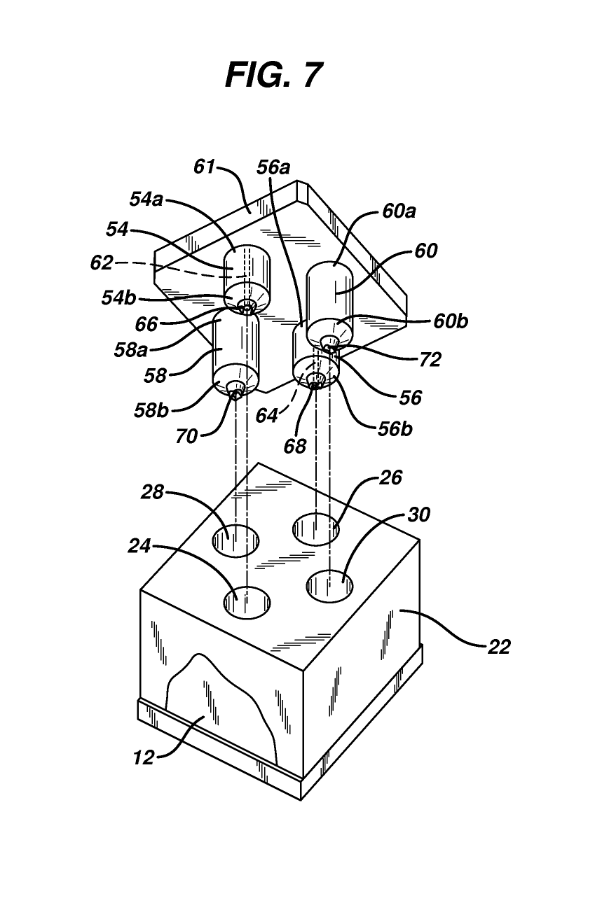

Referring to FIG. 7, a first set of plugs 54, 56, 58, 60 is arranged and configured for insertion into the ports 24, 26, 28, 30 to deliver fluid overmold material into the first overmold cavity 34 (again defined by the primary male mold half 12, the secondary female mold half 22, and the device frame 18, shown in FIGS. 4A, 4B). Each plug 54, 56, 58, 60 has a proximal end 54a, 56a, 58a, 60a mounted on a first plug mounting plate 61 and operatively connected to a source of fluid overmold material. Each plug 54, 56, 58, 60 has a distal end 54b, 56b, 58b, 60b disposed opposite thereof. Plugs 54, 56 correspond to the first pair of ports 24, 26. Each of plugs 54, 56 includes a passage 62, 64 therethrough to deliver the fluid overmold material through an aperture 66, 68 in its distal end and into the first overmold cavity 34. Plugs 58, 60 correspond to the second pair of ports 28, 30. Instead of apertures at their distal ends, each of plugs 58, 60 has a clamping surface 70, 72 that projects into the first overmold 34 and securely engages the device frame 18 disposed therein. In the view of FIG. 7, the first set of plugs 54, 56, 58, 60 and first plug mounting plate 61 are shown rotated towards the viewer in order to show the features of the distal end 54b, 56b, 58b, 60b of the plugs. In actual operation, the first set of plugs 54, 56, 58, 60 and first plug mounting plate 61 would be aligned for insertion into the ports 24, 26, 28, 30.

The clamping surfaces 70, 72 securely hold the device frame 18 in position as it is subjected to high pressure injection of the fluid overmold material into the mold cavity. Otherwise, the fluid material may flow between the device frame 18 and the primary male mold half 12 and provide uneven application of the overmold material.

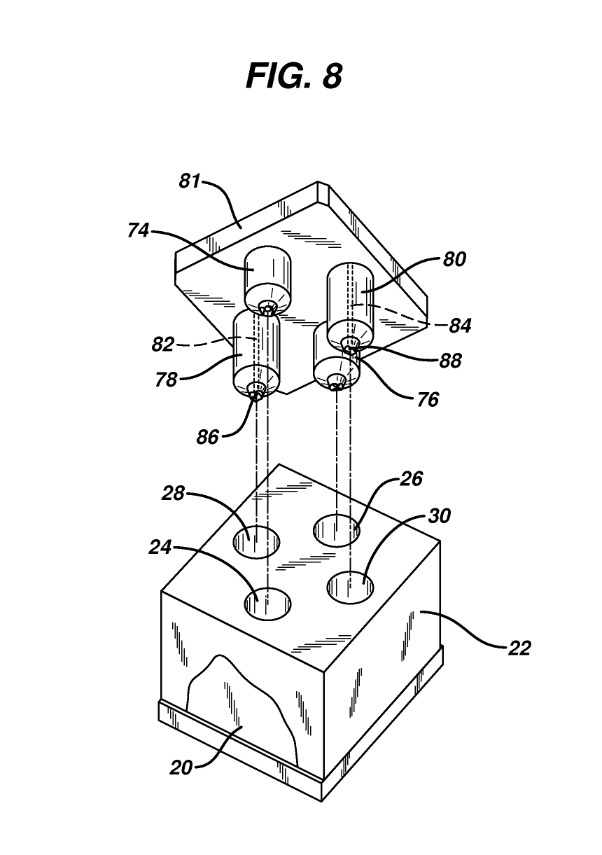

FIG. 8 shows the two-part mold 10 as reconfigured for the second overmold material injection step (also as shown in FIG. 5). A second set of plugs 74, 76, 78, 80 is arranged and configured for insertion into the ports 24, 26, 28, 30, and the proximal end of each plug is mounted on a second plug mounting plate 81. Plugs 74, 76 are inserted into the first pair of ports 24, 26. In this step, plugs 74, 76 do not include a passage therethrough. However, plugs 78, 80 include a passage therethrough to deliver the fluid overmold material through an aperture 86, 88 in a distal end of each plug and into the second overmold cavity 38. In FIG. 8 (similar to the view of FIG. 7), the second set of plugs 74, 76, 78, 80 and second plug mounting plate 81 are shown rotated towards the viewer in order to show the features of the distal end of the plugs. In actual operation, the second set of plugs 74, 76, 78, 80 and second plug mounting plate 81 also would be aligned for insertion into the ports 24, 26, 28, 30.

While the male and female mold halves are shown as unitary structures, it will be recognized that each mold half may be composed of two or more pieces that together form the respective mold half.

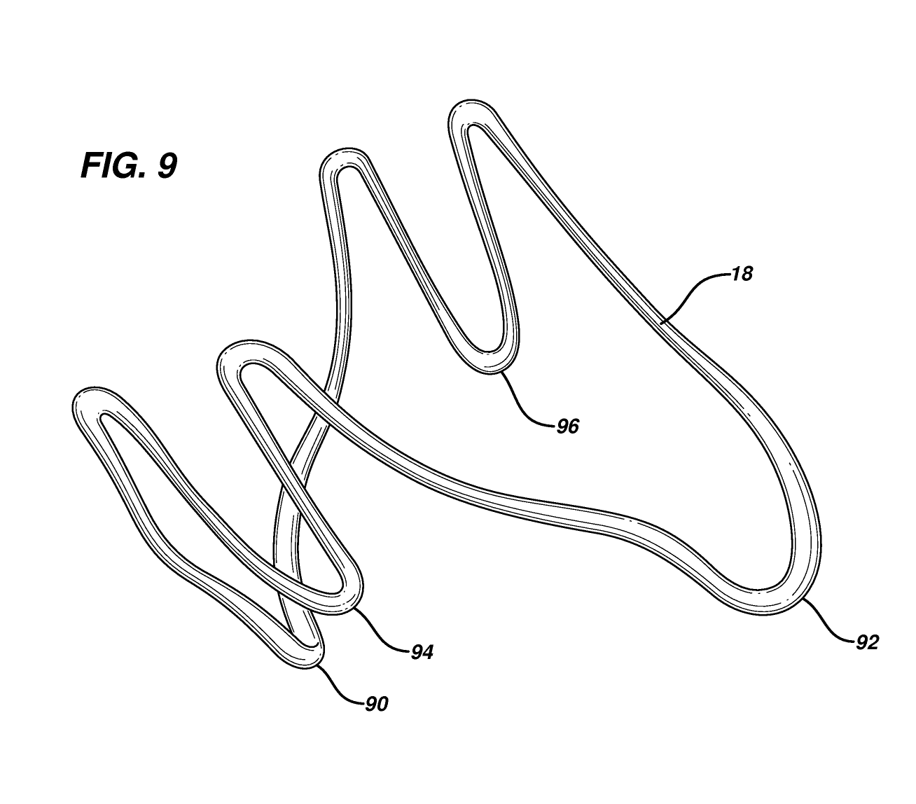

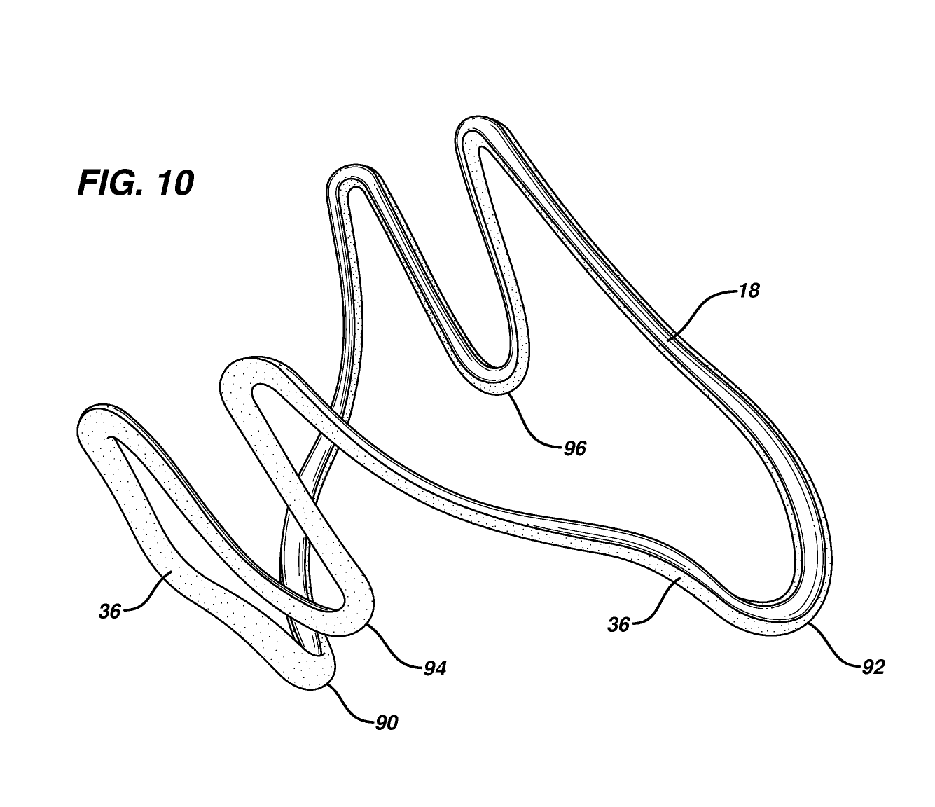

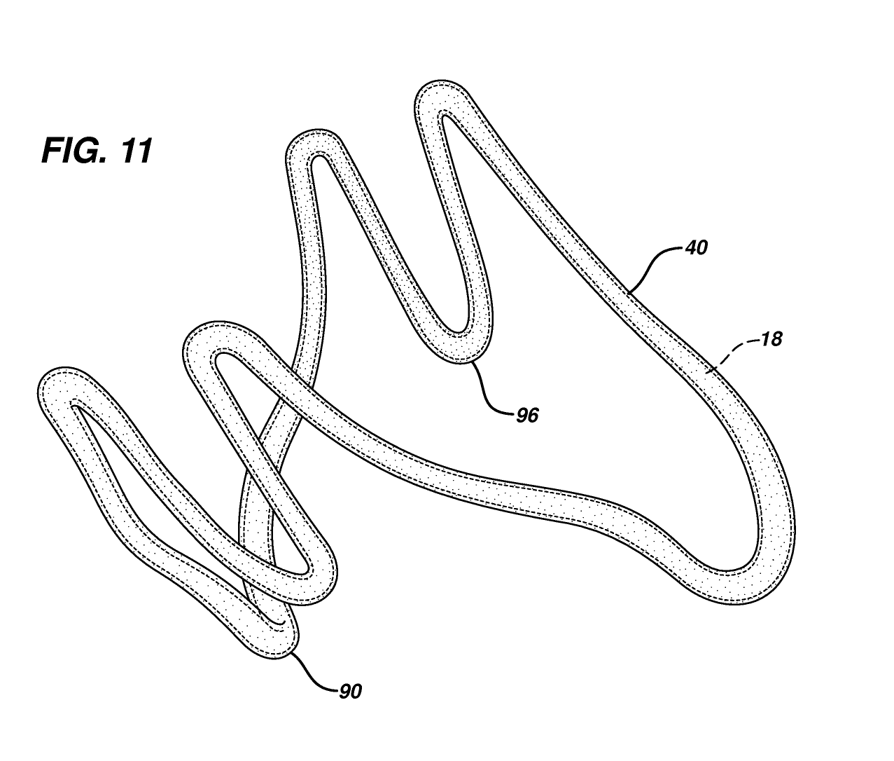

FIGS. 9-11 show the results of the molding steps discussed above for an incontinence device of the present invention. FIG. 9 shows the device frame 18, removed from the primary mold cavity 16. The device frame 18 is inverted from the orientation it would have in FIG. 6. In other words, anchoring curves 90, 92 of the device frame 18 corresponding to the upper mold cavity curves 46, 48 are shown at the bottom of FIG. 9, and inner peak curves 94, 96 of the “W-shaped” section of the working portion are directed toward the bottom of FIG. 9. FIG. 10 shows the device frame 18 of FIG. 9 with the outer layer portion 36 resulting from operation of the mold configuration of FIGS. 4 and 7. Because the device frame 18 is held in the clamping surface 70, 72 of plugs 58, 60 projecting into the first overmold 34, the inner peak curves 94, 96 of the “W-shaped” section of the working portion are not covered with the first outer layer portion 36. FIG. 11 shows the full outer layer portion 40 molded about the device frame 18 of FIG. 9, corresponding to the product of the mold configuration of FIGS. 5 and 8.

As noted above, working portion 9 of the intravaginal incontinence device includes a device frame 6 formed of a first structural material that provides resistance to compression and recovers from compression with sufficient force to provide the desired incontinence support. Useful structural materials are elastic or even superelastic materials. These structural materials include metals (including without limitation metal alloys), polymers (including without limitation shape memory polymers and high modulus polymers), composites of one or more polymers and/or filled or reinforced polymers, and combinations thereof. Shape memory materials include those disclosed in US Pat. App. Nos. 2008/0009664, and 2008/0033230, and 2008/0009662. High modulus polymers include those disclosed in copending application, Serial No. 12/645,800, filed on Dec. 23, 2009, entitled “Intravaginal Incontinence Device,” and Ser. No. 12/974,378, filed on Dec. 21, 2010, also entitled “Intravaginal Incontinence Device.”

Preferred high modulus polymers have an elongation at yield of at least 3% and an elastic modulus of at least 2 Gpa. A representative, non-limiting list of suitable high modulus polymers includes polyetherimide, polyetheretherketone, polycarbonate, co-polymers, specialized and/or modified plastics, filled plastics, and the like, that can provide these high modulus properties. Preferred high modulus polymers include polyetherimides and polyetheretherketones. These materials are further described in the above-mentioned copending application, Ser. No. 12/645,800, filed on Dec. 23, 2009, the contents of which are herein incorporated by reference.

FIG. 2 shows the device frame 6 without the cushioning material. The working portion 9 of the device frame 6 is formed of a plurality of connected elongate elements 9a. The elongate elements 9a that make up the working portion may directly or indirectly connect to those elongate elements that make up the anchoring portion 8. The working pressure exerted by the working portion 9 is determined by the material selected for the device frame 6 and by the dimensions and arrangement of the elongate elements that make up this device frame 6. Thicker elongate elements and/or shorter elongate elements can generally provide greater working pressures as these are capable of providing greater resistance to deformation of the device and, thus, greater expansion force when the device is compressed or reduced in cross-section. In addition, the angle between the elongate elements also influences the working pressure.

The elongate elements have a small cross-section in order to fit into a delivery applicator and to be comfortable for the user. The elongate elements should have a maximum linear cross-section dimension of less than about 5 mm, preferably, less than about 4 mm, and most preferably, less than about 3 mm. The elongate elements can have any useful cross-section shape, including without limitation, round, oval, elliptical, triangular, rectangular, etc. As one of ordinary skill will recognize, the change cross-section shape may provide various desired resilience, increased surface area for a given cross-sectional area, reduced material stress, and the like.

Anchoring portion may be formed of the same materials as the working portion, and in a preferred embodiment, both the working portion and the anchoring portion are formed of the same material in a unitary construction.

As shown in FIGS. 2 and 9-11, the device interior is preferably open, and the device frame 6 loosely defines this cavity or hollow. The bearing surfaces are generally disposed on outwardly-facing surfaces of the device frame 6.

As discussed above, the device frame 6 is overmolded with an outer layer 7 of cushioning material (as shown in FIGS. 1 and 2). This provides useful characteristics to the device. The cushioning material may provide one or more of the following properties to the intravaginal incontinence device: resiliency, shock absorbing, softness, elasticity, tear-resistance, protection of the frame from chemical degradation (for example, by oxidation or other chemical attack, especially in high stress portions), and the like. In addition, the cushioning material may provide other functions including acting as a carrier for medications, lotions, fragrances, odor neutralizers, lubricants, and the like. The cushioning material can also improve the aesthetics of the device, especially if the incontinence device is visible, and it can improve the ability of the device to stay in place by providing a textured and/or a more compliant surface.

The properties such as resiliency, shock absorbing, softness, elasticity, flexibility, and the like can provide the softness and cushioning to minimize excessive pressure on the vaginal tissues. Properties, such as elasticity and tear-resistance can provide additional safety in the event of breakage of the device frame. The cushioning material can act to contain such broken elements. In addition, the relatively soft, elastic, and/or flexible materials provide decrease the likelihood that parting lines from the molded part are sharp enough to be a source for irritation of vaginal tissues during insertion, use, and withdrawal of the device.

The cushioning material may be formed of any soft and/or flexible material useful in injection molding and/or dip molding processes that providing desired properties, such as thermoplastic elastomers. Useful materials for the cushioning material include, without limitation, urethanes, polyolefins (including polyethylenes, polypropylenes, ethylene-propylene diene monomers, etc.), co-polymers (including styrene-ethylene-butylene-styrene block co-polymers such as the KRATON® thermoplastic elastomers from Kraton Polymers), styrene acrylate co-polymers, silicones, rubber, latex, fibers, and the like. In addition, mixtures and blends of materials can also be used including, without limitation, Santoprene™ thermoplastic elastomer from ExxonMobil Chemical.

One measure of the appropriateness of the cushioning material is a measure of the Shore A Hardness. Preferably, the cushioning material has a Shore A Hardness of between about 0 to about 120, preferably in a range of about 20 to about 100, more preferably in a range of about 40 to about 90 Shore A Hardness.

As shown in FIG. 1, the device 1 may also be enclosed in a flexible bag 4 or other relatively loose cover. This bag may provide one or more beneficial properties. It may reduce friction between the intravaginal incontinence device and its applicator and/or vaginal tissue during deployment. The flexible bag 4 may hide or otherwise disguise the appearance of the device frame from view for a more acceptable consumer device. The flexible bag 4 may help control the device during insertion and removal. It may help the device to stay in place. The flexible bag 4 may also contain other optional components such as a suppository substance. Finally, the flexible bag 4 may increase the contact area for applying pressure to the bladder neck. The cover may also provide increased friction against the vaginal epithelium to help the device stay in place during use. Any medically appropriate materials may be used to form the bag, and depending upon the desired end-use, it may be opaque, light, and/or breathable. Useful bag materials include those used in the manufacture of tampons, such as nonwoven fabrics and plastic film, including apertured films. The bag itself may also be apertured.

The intravaginal incontinence device preferably includes a withdrawal element such as a removal string 5. This may be crisscrossed between the elongate elements of the device frame to create a “cinch sac” mechanism. Any string or cord known in the sanitary protection art may be useful for this purpose. As the strings are pulled during removal, the elongate elements are gathered together to create a smaller diameter device during removal. Cinching the device at its base may make removal of the device more comfortable and easier as it makes the diameter of the device smaller and the shape conducive to remove easily.

The intravaginal incontinence device may be contained within an applicator similar to those known for use in delivering tampons and suppositories. The applicator may be a push-type applicator or a retractable applicator. Preferred delivery applicators have a maximum internal diameter of less than about 24 mm, more preferably, less than about 19 mm, and most preferably less than 16 mm. A collar may be added to control the depth of insertion.

In one preferred embodiment, the cushioning material is non-uniform about the device frame 6 that it covers. We have found that biasing the cushioning material to the outer surfaces of the device frame may provide more useful cushioning while minimizing the volume of the cushioning material that merely adds bulk to the device.

What is claimed is:

1. A method of making an injection molded device comprising the steps of:
a. assembling a mold comprising a primary male mold half and a primary female mold half to define a primary mold cavity;
b. injecting a first fluid polymeric material into the primary mold cavity and permitting the first fluid polymeric material to solidify to form a device frame;
c. removing the primary female mold half and replacing it with a secondary female mold half to provide a first overmold cavity defined by the primary male mold half, the secondary female mold half, and the device frame;
d. restraining the device frame in the first overmold cavity and injecting a second fluid polymeric material into the first overmold cavity and permitting the second fluid polymeric material to solidify to sufficiently to form a first portion of an outer layer that retains the shape of the first overmold cavity;
e. removing the primary male mold half and replacing it with a secondary male mold half to provide a second overmold cavity defined by the secondary male mold half, the secondary female mold half, the device frame, and the first portion of the outer layer;
f. injecting an additional amount of the second fluid polymeric material into the second overmold cavity and permitting the second fluid polymeric material to unite with the first portion of the outer layer to form a sufficiently integral outer layer about the device frame.

2. The method of claim 1 wherein the male and female mold halves are unitary structures.

3. The method of claim 1 wherein at least one of the male and female mold halves comprise a plurality of pieces that together form such mold half.

4. A method of making an injection molded intravaginal urinary incontinence device comprising the steps of:
a. assembling a mold comprising a primary male mold half and a primary female mold half to define a primary mold cavity;
b. injecting a fluid high modulus polymeric material into the primary mold cavity and permitting the high modulus polymeric material to solidify to form a device frame;
c. removing the primary female mold half and replacing it with a secondary female mold half to provide a first overmold cavity defined by the primary male mold half, the secondary female mold half, and the device frame;
d. restraining the device frame in the first overmold cavity and injecting a fluid cushioning polymeric material into the first overmold cavity and permitting the cushioning polymeric material to solidify to sufficiently to form a first portion of an outer layer that retains the shape of the first overmold cavity;
e. removing the primary male mold half and replacing it with a secondary male mold half to provide a second overmold cavity defined by the secondary male mold half, the secondary female mold half, the device frame, and the first portion of the outer layer;
f. injecting an additional amount of the fluid cushioning polymeric material into the second overmold cavity and permitting the cushioning polymeric material to unite with the first portion of the outer layer to form a sufficiently integral outer layer about the device frame.

5. The method of claim 4, further comprising the step of enclosing the intravaginal urinary incontinence device in a flexible bag.

6. The method of claim 4, further comprising the step of attaching a withdrawal element to the intravaginal urinary incontinence device.

7. The method of claim 6, wherein the withdrawal element is attached to one or more elongate elements of the device frame.

8. The method of claim 4, wherein the biocompatible material comprises one or more thermoplastic elastomers.

9. The method of claim 4 wherein the male and female mold halves are unitary structures.

10. The method of claim 4 wherein at least one of the male and female mold halves comprise a plurality of pieces that together form such mold half.

11. The intravaginal urinary incontinence device formed by the method of claim 4.
